(19) United States
(12) Patent Application Publication
(10) Pub. No.: US 2022/0379006 A1
(43) Pub. Date: Dec. 1, 2022

(54) PRIMARY CONTAINER ASSEMBLY WITH INTEGRATED FLUID PATH

(71) Applicant: WEST PHARMACEUTICAL SERVICES, INC., Exton, PA (US)

(72) Inventors: Tommy Gene DAVIS, Athens, TX (US); Rohit VORA, Exton, PA (US); Ran HEZKIAHU, Herzliya (IL)

(21) Appl. No.: 17/791,574

(22) PCT Filed: Jan. 12, 2021
(86) PCT No.: PCT/US2021/013017

§ 371 Date: Jul. 8, 2022

Related U.S. Application Data
(60) Provisional application No. 62/961,933, filed on Jan. 16, 2020.

(51) Int. Cl. A61M 5/00; A61M 5/31; A61M 5/32; A61M 5/34; A61M 25/00

(52) U.S. Cl. CPC A61M 5/002; A61M 5/3134; A61M 5/3202; A61M 5/345; A61M 25/002; A61M 2025/0091; A61M 2207/00

(57) ABSTRACT

A primary container assembly includes a primary container defining a body having a proximal end defining an opening configured to receive a plunger, a distal end opposite the proximal end and defining an outlet, and a chamber extending from the proximal end to the distal end that is configured to receive a drug. The primary container assembly further comprises a tube extending from a first end that is integrally attached to the distal end of the primary container to a second end opposite the first end, where the tube defines a channel extending from the first end to the second end. The primary container assembly also includes a hollow needle configured to penetrate skin of a patient.

### CROSS-REFERENCE TO RELATED APPLICATIONS

This application claims the benefit of U.S. Provisional Patent App. No. 62/961,933, filed Jan. 16, 2020, the entire contents of which are hereby incorporated by reference as if set forth in its entirety herein.

### TECHNICAL FIELD

This application is directed to a primary container assembly for storing and dispensing a drug. Specifically, this application relates to a primary container assembly for ensuring sterility of a fluid path from manufacturing, through introduction of the drug in the primary container assembly, and to the end user.

### BACKGROUND

In order to dispense a drug into the subcutaneous tissue, intramuscular tissue, or veins of a patient, a fluid path is required to allow the drug to flow out of a primary container storing the drug and through a needle. As a result, an interface is required between the primary container, fluid path, and needle. To form such a connection, conventional primary containers can have an adaptive end (septum, Luer lock) or other interfacing features. However, when interfacing the primary container with the fluid path, needle, or other components of a dispensing system, sterility of the primary container assembly can be compromised. For example, when interfacing a primary container, fluid path, and needle, the connection point between each of these components can have compromised sterility during or after connection, thus leading to a loss of sterility of the system. This can necessitate swabbing of various fittings with alcohol wipes prior to connection to mitigate or minimize risk, leading to greater process complexity. Additionally, such interfacing efforts often require an arrangement of several unique and specially designed interface features that further increase the components through which sterility can be compromised, as well as increase costs and complexity associated with assembling a dispensing device with the goal of ensuring end-to-end sterility.

As a result, there is a need for a primary container assembly having decreased complexity that ensures end-to-end sterility in a drug loading and dispensing process.

### SUMMARY

An embodiment of the present disclosure is a primary container assembly comprising a primary container defining a body having a proximal end defining an opening configured to receive a plunger, a distal end opposite the proximal end and defining an outlet, and a chamber extending from the proximal end to the distal end that is configured to receive a drug. The primary container assembly further comprises a tube extending from a first end that is integrally attached to the distal end of the primary container to a second end opposite the first end, where the tube defines a channel extending from the first end to the second end. The primary container assembly also comprises a hollow needle configured to penetrate skin of a patient, wherein the hollow needle is integrally attached to the second end of the tube, where the tube is configured to direct the drug from the chamber of the primary container to the needle.

A further embodiment of the present disclosure is a method for assembling a primary container assembly. The method includes providing a primary container defining a body having a proximal end defining an opening configured to receive a plunger, a distal end opposite the proximal end and defining an outlet, and a chamber extending from the proximal end to the distal end that is configured to receive a drug. The method further includes attaching a first end of a tube to the distal end of the primary container, such that the tube is in fluid communication with the outlet, and attaching a hollow needle to a second end of the tube.

### BRIEF DESCRIPTION OF THE DRAWINGS

The foregoing summary, as well as the following detailed description, will be better understood when read in conjunction with the appended drawings. The drawings show illustrative embodiments of the disclosure. It should be understood, however, that the application is not limited to the precise arrangements and instrumentalities shown.

FIG. 1 is an exploded view of a primary container assembly according to an embodiment of the present disclosure;

FIG. 2 is a side view of the primary container and a tube of the primary container assembly shown in FIG. 1, where the tube is directly attached to the primary container;

FIG. 3 is a side view of the primary container and tube of the primary container assembly shown in FIG. 1, where the tube is attached to the primary container via a needle;

FIG. 4 is a perspective view of the tube of the primary container assembly shown in FIG. 1 in an uncoiled configuration;

FIG. 5A is a perspective view of a needle hub and needle of the primary container assembly according to an embodiment of the present disclosure;

FIG. 5B is a perspective view of a needle hub and needle shield of the primary container assembly according to a further embodiment of the present disclosure;

FIG. 6A is a cross-sectional view of the needle hub and needle shown in FIG. 5A;

FIG. 6B is a cross-sectional view of the needle hub and needle shown in FIG. 5A, with a needle shield attached.

FIG. 7A is a cross-sectional view of the primary container assembly shown in FIG. 1, with a single cup attached to the primary container;

FIG. 7B is a cross-sectional view of the primary container assembly shown in FIG. 1, with two cups attached to the primary container;

FIG. 8 is a schematic view of components of a primary container assembly according to the present disclosure installed within a drug dispensing device;

FIG. 9 is a process flow diagram of a method of assembling a primary container assembly according to an embodiment of the present disclosure;

FIG. 10A is a perspective view of the primary container assembly shown in FIG. 7B in combination with a compatible nest assembly; and

FIG. 10B is a side cross-sectional view of the primary container assembly and nest assembly shown in FIG. 10A, with the primary container assembly supported in a filling position.

### DETAILED DESCRIPTION OF ILLUSTRATIVE EMBODIMENTS

Described herein are primary container assemblies 10, 10′ for storing and dispensing a supply of a drug. Certain terminology is used to describe the primary container assemblies 10, 10′ in the following description for convenience only and is not limiting. The words “right”, “left”, “lower,” and “upper” designate directions in the drawings to which reference is made. The words “inner” and “outer” refer to directions toward and away from, respectively, the geometric center of the description to describe primary container assemblies 10, 10′ and related parts thereof. The words “forward,” “rearward,” “proximal,” and “distal” refer to directions toward or away the proximal or distal ends of body of the component of the primary container assemblies 10, 10′ being referred to. The terminology includes the above-listed words, derivatives thereof and words of similar import.

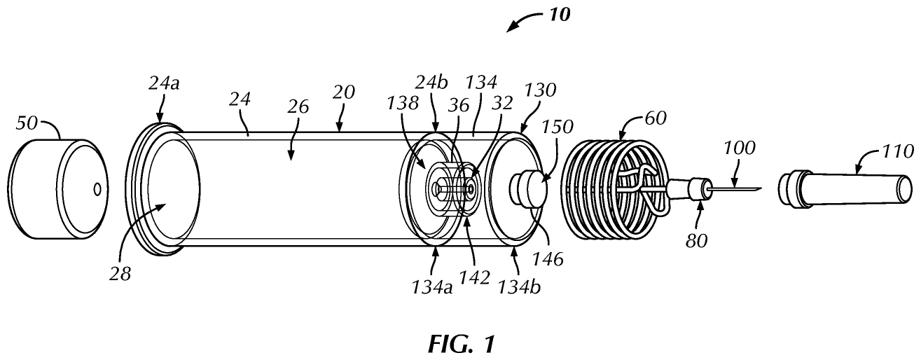

An embodiment of the present disclosure comprises a primary container assembly 10 configured to store and deliver a supply of a drug. The primary container assembly 10 can include a primary container 20, plunger 50, tube 60, needle 72, needle hub 80, needle 100, needle shield 110, cup 130, and/or second cup 160, each of which will be described in detail below. Referring to FIGS. 1-3, the primary container 20 can define a body 24 that extends from a proximal end 24a to a distal end 24b opposite the proximal end 24a, where the body 24 can be comprised of a plastic, such as cyclic olefin or other medical grade plastic, or glass. The body 24 can define a substantially elongate, cylindrical shape, though other shapes are contemplated. In one embodiment, the primary container 20 is configured as a syringe as described in International Patent Application No. PCT/US2015/049588, assigned to West Pharmaceutical Services, Inc. of Exton, Pa., though the present disclosure is not intended to be limited to such. The body 24 of the primary container 20 further defines a chamber 26 extending from the proximal end 24a to the distal end 24b, where the chamber 26 is configured to receive and store a supply of a drug, and from which the drug is ultimately dispensed, as will be described below.

The proximal end 24a of the primary container 20 can define a flange to enable easier grasping or ensure proper positioning of the primary container 20 within a dispensing device. The proximal end 24a can also define an opening 28 configured to provide access to the chamber 26. Likewise, the distal end 24b can define an outlet 32 configured to further provide access to the chamber 26. The opening 28 can define a diameter that is larger than a diameter of the outlet 32, such that the opening 28 is configured to receive a plunger 50. In operation, the chamber 26 can be filled from a drug source (not shown) through the opening 28. The plunger 50 can be disposed within the chamber 26 after the chamber 26 has been filled so as to prevent drug from leaking out of the chamber 26 through the opening 28. Further, in operation the plunger 50 can be translated distally through the chamber 26 while sealingly engaging the inner surface of the body 24 so as to force the drug from the chamber 26 through the outlet 32 while maintain a fluid seal with the primary container 20. The plunger 50 can comprise a conventional rubber or plastic plunger, though other embodiments are contemplated. The plunger 50 can be operably attached to an actuation mechanism (not shown) for selectively translating the plunger 50 through the chamber 26, where the actuation mechanism can comprise a motor, spring, pneumatic device, telescoping assembly, or other means capable of causing linear actuation. In one embodiment, the distal end 24b of the primary container 20 comprises a Luer connection, though the distal end 24b can include any structure capable of interfacing with a length of tube 60, which will be described further below.

Referring to FIGS. 1-5B, the tube 60 of the primary container assembly 10 will be described in greater detail. The tube 60 can have a body 64 that extends from a first end 64a to a second end 64b opposite the first end 64a. The body 64 can define a channel 68 extending therethrough from the first end 64a to the second end 64b, such that the body 64 is substantially configured as a hollow tube. The body 64 can be comprised of a medical grade plastic, metal, such as steel or nitinol, etc., though the present disclosure is not intended to be limited to such. In one embodiment, the body 64 is substantially rigid, and as a result defines a fixed shape. In another embodiment, the body 64 is flexible, and as a result can be reconfigured and reshaped by a manufacturer or user as desired. For example, the tube 60 can be coiled during initial packaging and uncoiled during end use. To place the tube 60 in the coiled configuration, the tube 60 can be sequentially wrapped around an extension 36 of the primary container 20 so as to cause at least a portion of the tube 60 to take a coiled configuration comprising a plurality of axially arranged coils. A cup 130 can be placed over the coiled portion of the tube 60 to secure the tube 60 in the coiled configuration, as will be described below. However, in other embodiments the cup 130 can also comprise a portion of the body 24 of the primary container 20 (molded together or as an attachment).

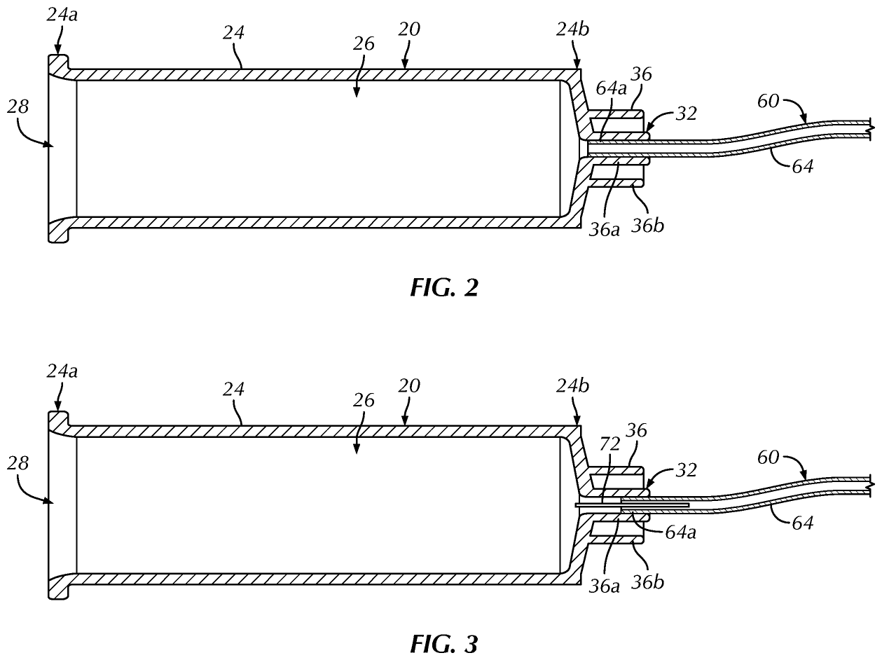

As shown in FIGS. 2 and 3, the first end 64a of the tube 60 can be integrally attached to the distal end 24b of the primary container 20. Specifically, the distal end 24b of the primary container 20 can include an extension 36 extending distally therefrom, where the extension 36 can comprise one or more concentrically arranged hollow cylindrical necks. For example, in the depicted embodiment the extension 36 comprises an inner neck 36a and an outer neck 36b concentrically positioned around the inner neck 36a, though other embodiments are contemplated. The inner neck 36a of the extension 36 can define the portion of the distal end 24b that defines the outlet 32. In one embodiment (FIG. 2), the extension 36 of the primary container 20 can be molded over the first end 64a of the tube 60. In another embodiment, the first end 64a of the tube 60 can be integrally attached to a Luer connection (not shown) of the primary container 20. Optionally, the extension 36 of the primary container 20 can be molded over a portion of a needle 72 (FIG. 3), where the first end 64a of the tube 60 is configured to engage another portion of the needle 72. As a result, the needle 72 can define an interface between the primary container 20 and the tube 60. It is contemplated that the tube 60 can be irremovably attached to the primary container 20. It is also contemplated that the primary container 20 and tube 60 can be formed such that the primary container 20 and tube 60 define a one-piece, monolithic structure, such as through co-injection molding. In another embodiment, an interface component (not shown) can be attached to the first end 64a of the tube 60, where the interface component is configured to snap onto or frictionally engage the distal end 24b of the primary container 20.

The second end 64b of the tube 60 can be integrally attached to a needle hub 80 that defines an interface between a hollow needle 100 and the tube 60. Though depicted as a hollow needle, it is contemplated that in other embodiments the needle 100 can be replaced with a rigid or soft cannula. The needle 100 can comprise an elongate, hollow metal needle, though other configurations are contemplated. For example, the body of the needle 100 can define a 90-degree angle in some embodiments, or any other angle desired. The needle 100 can define a body 104 that extends from a first end 104a to a second end 104b opposite the first end 104a. The second end 104b of the hollow needle 100 can be configured to penetrate skin of a patient, i.e., administer the drug from the chamber 26 of the primary container 20 to the patient. As such, the second end 104b can define a sharp tip. Whereas the primary container 20 is attached to the first end 64a of the tube 60, the first end 104a of the needle 100 is attached to the second end 64b of the tube 60. Due to the inclusion of the tube 60, in this configuration the number of materials used to create the fluid path is minimized, thus eliminating multiple adhesive joints. Further, this design provides the ability to place the needle 100 anywhere on the delivery device without any structural constraints related to the primary container 20.

The primary container assembly 10 can further include a needle hub 80 attached to the needle 100. The needle hub 80 can have a body 82 defining an outer surface 82a, as well as a channel 84 extending therethrough. The second end 64b of the tube 60 is configured to be secured at least partially within the channel 84 of the needle hub 80. Additionally, the hollow needle 100 is configured to be secured at least partially within the channel 84. It is contemplated that in some embodiments, the needle 100 can be at least partially received within the channel 68 of the tube 60.

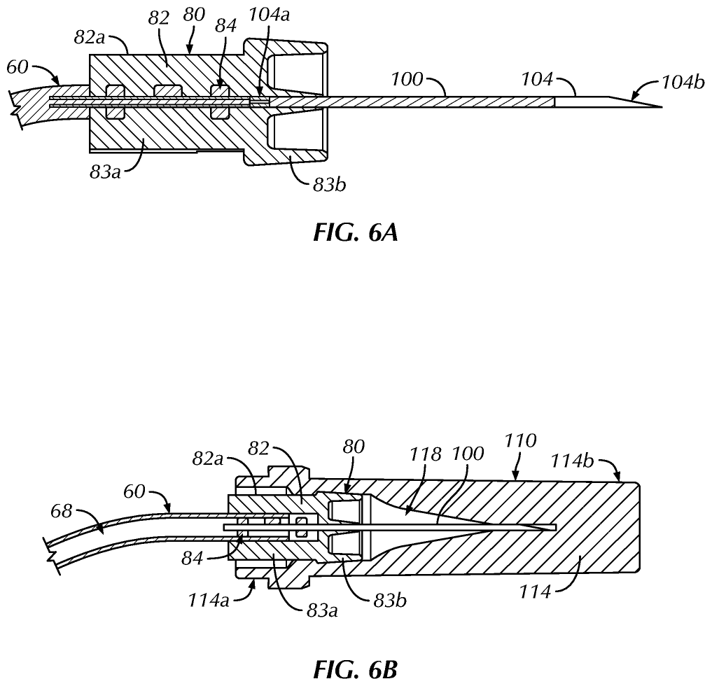

A first embodiment of a needle hub 80 is shown in FIGS. 5A, 6A, and 6B, while a second embodiment of a needle hub 80′ is shown in FIG. 5B. The body 82 of the needle hub 80 can extend substantially coaxially along a singular axis. Further, a portion of the body 82 can be frustoconical in shape, the function of which will be described below. For example, the body 82 of the needle hub 80 can define a first section 83a at the proximal end of the body 82, and a second section 83b extending distally from the first section 83a. The first section 83a can define a substantially elongate tube, while the second section 83b can have a frustoconical shape. In contrast, the needle hub 80′ can have a portion that extends at substantially a 90-degree angle relative to another portion of the needle hub 80′. It is contemplated that the needle hubs 80, 80′ can be irremovably attached to the tube 60. The different shapes of the needle hubs 80, 80′ can be configured to create particular interferences with primary container assembly components or components of the device within which the primary container assembly 10, 10′ is to be received.

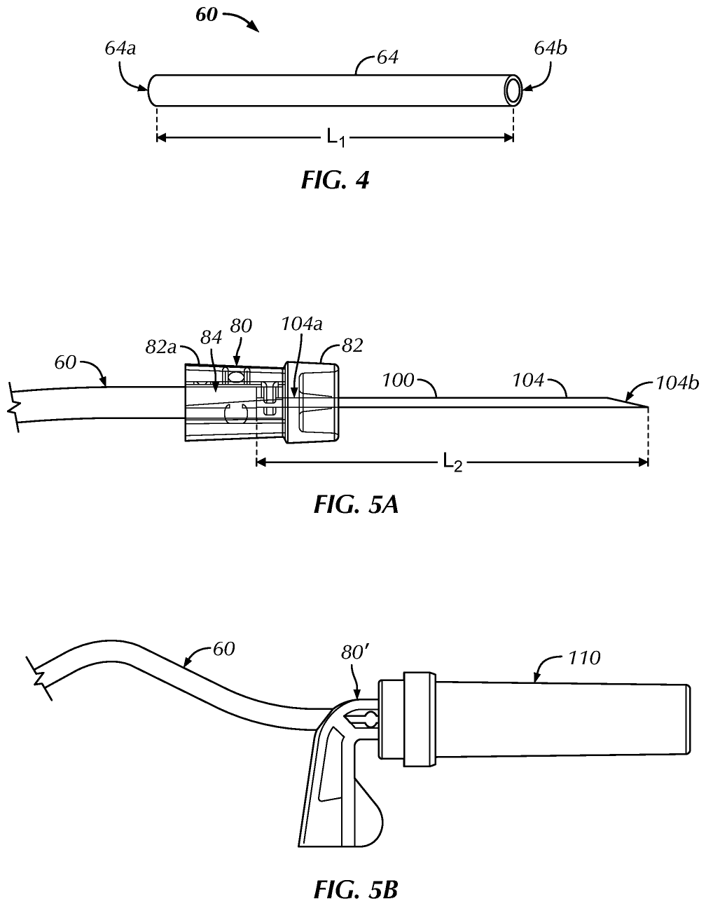

As shown in FIG. 4, the tube 60 can define a first length L₁ measured along an axis coaxial with the body 64 of the tube 60 from the first end 64a to the second end 64b. Likewise, the needle 100 can define a second length L₂ measured from its base to its skinpiercing tip, where the first length L₁ is greater than the second length L₂. Optionally, the first length L₁ can be two times, three time, four times, etc. greater than the second length L₂.

As described above, the primary container assembly 10 is configured to cause, under force applied by the plunger 50, the drug to flow from the chamber 26 of the primary container 20, through the tube 60, through the needle hub 80 and needle 100, and into the patient. The configuration of the primary container 20, tube 60, and needle 100 as an integral assembly creates ensured sterility of the drug environment throughout transportation and initial setup of the primary container assembly 10 at the end use site. In conventional primary containers, the primary container must be fluidly attached to other components of a dispensing system through complicated and time intensive processes at the end use site, potentially compromising the sterility of the fluid pathway. The primary container assembly 10 provides a singular fluid path with greatly increased risks for compromised sterility.

To further ensure fluid pathway sterility and prevent unintended injury from contact with the needle 100, the primary container assembly 10 can include a needle shield 110 disposed over at least a portion of the needle 100 and releasably connected to the needle hub 80. Referring to FIG. 6B, the needle shield 110 can have a body 114 that extends from a first end 114a to a second end 114b opposite the first end 114a. It is contemplated that the needle shield 110 can be comprised of a soft or rigid material. For example, the body 114 of the needle shield 110 can be comprised of rubber, though other types of materials are contemplated. The needle shield 110 can define a cavity 118 extending into the body 114 from the first end 114a, where the cavity 118 terminates at a location axially between the first and second ends 114a, 114b. In operation, the cavity 118 can be sized to receive a portion of the needle 100 and at least a portion of the needle hub 80 so as to cover the needle 100, thus ensuring sterility of the needle 100 and preventing injury from human contact with the second end 104b of the needle 100.

When disposed within the cavity 118, a portion of the needle hub 80 can frictionally engage the needle shield 110 so as to secure the needle shield 110 to the needle hub 80. For example, the second section 83b (the frustoconical portion) can frictionally engage the needle hub 80. When the needle hub 80 is inserted into the cavity 118, a progressively increasing diameter of the second section 83b of the needle hub 80 can come into contact with the needle shield 110, thus leading to an eventual frictional engagement with the needle shield 110. Additionally, the cavity 118 can be designed so as to ensure that a predetermined exposure length of the needle 100 is maintained throughout transportation of the needle assembly 10. By maintaining a predetermined length of the needle 100 in contact with the needle shield 110 through transportation, the primary container assembly 10 can ensure sterility of the needle 100 is maintained and the needle 100 does not repeatedly impact the needle shield 100 during transportation, which may otherwise create material fragments.

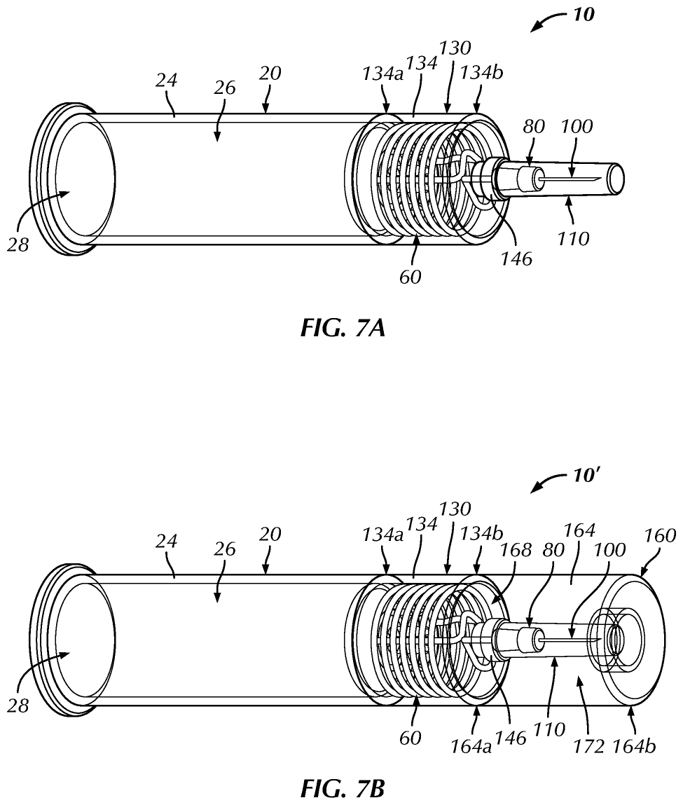

Referring to FIG. 7A, the primary container assembly 10 can further include a cup 130 attached to the distal end 24b of the primary container 20. The cup 130 can have a substantially hollow body 134 that extends from an open proximal end 134a to a substantially closed distal end 134b. Specifically, the proximal end 134a can define an opening 138, and the body 134 defines a cavity 142 extends into the body 134 from the opening 138. The proximal end 134a can be configured to releasably attach to the distal end 24b of the primary container 20. In one embodiment, the cup 130 can be frictionally attached to the primary container 20. However, it is contemplated that the cup 130 can be attached to the primary container 20 through a snap fit, threaded engagement, label applied or shrink-wrapped to the primary container 20 and cup 130, etc. When attached to the primary container 20, the cup 130 can be disposed at least partially over the tube 60 when the tube 60 is in the coiled configuration. As a result, when the tube 60 is placed in the coiled configuration, the tube 60 can be substantially received within the cavity 142 of the cup 130. This can function to keep the tube 60 in the coiled configuration throughout transportation until the primary container assembly 10 is unpackaged for filling or use.

Once the needle shield 110 is attached to the needle hub 80, the cup 130 can be utilized to secure the needle shield 110 to the primary container 20. As shown in FIG. 7A, the cup 130 can include an extension 146 extending from its distal end 134b, where the extension 146 defines a channel 150 extending therethrough. When the cup 130 is placed over the portion of the tube 60 in the coiled configuration and releasably attached to the primary container 20, the needle hub 80 and at least a portion of the tube 60 attached thereto can be fed through the channel 150 of the extension 146, at which point the needle shield 110 can be disposed over the needle hub 80 and the needle 100. The needle shield 110 can further be releasably secure to the cup 130, and in particular the extension 146 of the cup 130. The needle shield 110 can be releasably attached to the cup 130 through a snap fit, threaded engagement, label applied or shrink-wrapped to the needle shield 110 and cup 130, etc.

Once the primary container assembly 10 has reached the final assembly, the components can be unpackaged as follows. First, the needle shield 110 can be detached from the cup 130 and the needle hub 80. Then, the cup 130 can be detached from the primary container 20 and removed from placement over the tube 60 in the coiled configuration. At this point, the user can be free to transition the tube 60 from the coiled configuration to an uncoiled configuration, in which the tube 60 is uncoiled from around the distal end 24b of the primary container, particularly the extension 36 of the primary container 20.

Though the primary container assembly 10 is depicted and described as including the cup 130 for securing the tube 60 in the coiled configuration, it is contemplated that in other embodiments other devices can be utilized for this purpose. For example, it is contemplated that circumferential grooves (for example, spiraling threads) can be defined by the extension 36, where the tube 60 can be coiled around the extension 36 such that the tube 60 is fitted within the grooves to secure the tube 60 to the extension 36. In this or other configurations, the cup 130 may or may not be included.

FIG. 7A depicts a primary container assembly 10 including a single cup 130 utilized to secure the tube 60 in the coiled configuration and the needle shield 110 to the primary container 20. However, as shown in FIG. 7B, another embodiment of a primary container assembly 10′ is depicted that includes a second cup 160. The primary container assembly 10 has many similar features as the primary container assembly 10′, and such features will be similarly labeled and not described herein for brevity. The second cup 160 can have a body 164 defining an open first end 164a and a substantially closed second end 164b opposite the first end 164a. Specifically, the first end 164a can define an opening 168, and the body 164 defines a cavity 172 extends into the body 164 from the opening 168. The first end 164a can be configured to releasably attach to the distal end 134b of the cup 130. In one embodiment, the second cup 160 can be frictionally attached to the first cup 130. However, it is contemplated that the second cup 160 can be attached to the cup 130 through a snap fit, threaded engagement, label applied or shrink-wrapped to the cup 130 and second cup 160, etc. Alternatively, the cup 130 and second cup 160 can be formed as a monolithic body. In such an embodiment, the cup 130 and second cup 160 can be formed with breakable members at their interface for manual separation of the cup 130 and second cup 160.

When attached to the cup 130, the second cup 160 can be disposed at least partially over the needle shield 110 when the tube 60 is in the coiled configuration. Additionally, the second cup 160 can be disposed at least partially over the needle hub 80 and needle 100 when the needle 100 is received within the needle shield 110. The second cup 160 can function to secure the needle shield 110 during transport of the primary container assembly 10′ and offer further protection to the sterility of components of the primary container assembly 10′. Though embodiments of a primary container assembly 10, 10′ including cup 130 and/or second cup 160 are shown and described in relation to FIGS. 7A and 7B, it is contemplated that in other embodiments of a primary container assembly, no such cups may be included. One benefit of the embodiment shown in FIG. 7B is the symmetry of the primary container assembly 10′ about its central axis, which provides certain benefits during filling, handling, and inspection of the primary container 20. For example, the second cup 160 is configured to interface with a conventional filling machine.

The primary container assemblies 10, 10′ can be advantageous in that they define a relatively constant mass about their longitudinal central axis, which allows them to be filled and inspected in conventional ways. For example, the primary container assemblies 10, 10′ can be rotated at a high RPM during camera inspection of the drug contents. Such processes would be difficult with a non-symmetric mass around the longitudinal central axis, especially one that allows the needle to be biased away from the central axis line of the primary container.

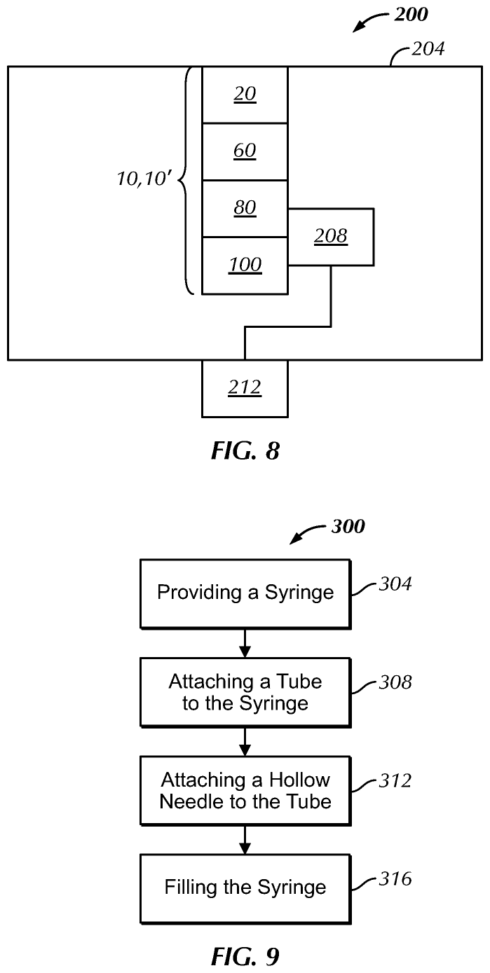

Referring to FIG. 8, a schematic diagram is depicted of a dispensing device 200 configured to dispense a drug. For example, the dispensing device 200 can be a wearable injection device, handheld injection device, or type of device capable of injecting a drug into a patient, though other types of devices are also contemplated. As shown in FIG. 8, the dispensing device 200 can be configured to utilize the primary container assembly 10, 10′ as the source for the drug. In one embodiment, the primary container assembly 10, 10′ can be manually loaded into the dispensing device 200 by the end user (after removing needle shield 110, cup 130, and/or second cup 160, as described above). Alternatively, the dispensing device 200 can be pre-loaded with components of the primary container assembly 10, 10′ by the manufacturer of the dispensing device 200.

The dispensing device 200 can include a body 204 configured to at least partially receive and secure components (such as the primary container 20, plunger 50, tube 60, and needle hub 80) of the primary container assembly 10, 10′. As such, the body 204 can be at least partially hollow. The dispensing device 200 can further include an input 208 configured to engage the needle hub 80 and/or needle 100 when components of the primary container assembly 10, 10′ are installed within the body 204 of the dispensing device 200. This engagement can be such that the tube 60 is in fluid communication with the input 208. The dispensing device 200 can also include an output component 212 in fluid communication with the input 208. The output component 212 can be a needle or cannula configured to pierce the skin of a patient, though other output components are contemplated. Alternatively, it is contemplated that the dispensing device 200 can include no output component 212, and rather the needle 100 of the primary container assembly 10, 10′ can function as the output component. In operation, when the components of the primary container assembly 10, 10′ are received within the dispensing device 200, the needle hub 80 and/or the needle 100 can releasably engage the input 208. For example, the engagement can comprise an interference fit, threaded engagement, snap-fit, etc., though other types of attachment are also contemplated. When the needle hub 80 and/or the needle 100 are engaged with the input 208, the dispensing device 200 can be configured to selectively dispense the drug from the primary container 20, through the tube 60, through the needle hub 80 and needle 100 and into the input 208, from the input 208 to the output component 212, and out of the output component 212 to the patient. A benefit of utilizing the primary container assembly 10, 10′ is that the dispensing device 200 does not require sterilization prior to attachment of the primary container assembly 10, 10′, as may be the case in other dispensing devices. This is because the fluid path defined by the primary container assembly 10, 10′ maintains sterility throughout assembly.

Referring to FIG. 9, a method 300 for assembly of the primary container assembly 10, 10′ is depicted. Method 300 can begin with step 304, which includes providing the primary container 20, where the primary container 20 defines a body 24 having a proximal end 24a defining an opening 28 configured to receive a plunger 50, a distal end 24b opposite the proximal end 24a and defining an outlet 32. The primary container 20 also defines a chamber 26 extending from the proximal end 24a to the distal end 24b and is configured to receive a drug. Step 308 can include attaching a first end 64a of the tube 60 to the distal end 24b of the primary container 20, such that the tube 60 is in fluid communication with the outlet 32. Step 308 can comprise molding the distal end 24b of the primary container 20 over the first end 64a of the tube 60. Step 308 can further comprise monolithically forming the tube 60 and the primary container 20. After step 308, in step 312 a hollow needle 100 can be attached to the second end 64b of the tube 60. Then, in step 316, the primary container 20 can be filled with the drug.

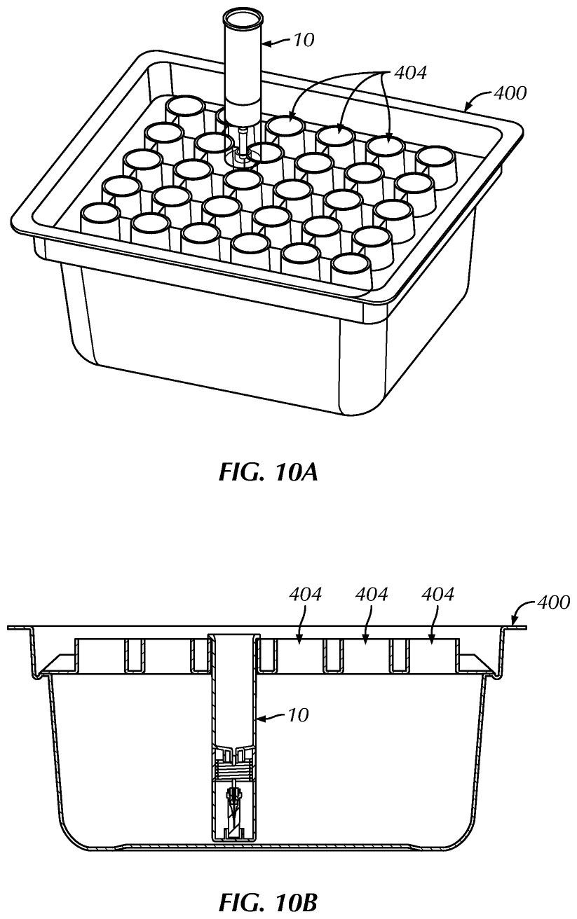

Referring to FIGS. 10A and 10B, the primary container assembly 10, 10′ can be configured to interface with a nest assembly 400. Specifically, the nest assembly 400 can define a plurality of apertures 404 configured to receive a respective one of the primary container assemblies 10, 10′. As shown particularly with respect to FIG. 10B, which a primary container assembly 10, 10′ is received by the nest assembly 400 in a position where the primary container assembly 10, 10′ is oriented for filling with a medicament, none of the tube 60, needle shield 110, cup 130, or other related components interfere with the nest assembly 400 or the orientation of the primary container assembly 10, 10′ generally.

While the invention is described herein using a limited number of embodiments, these specific embodiments are not intended to limit the scope of the invention as otherwise described and claimed herein. The precise arrangement of various elements and order of the steps of articles and methods described herein are not to be considered limiting. For instance, although the steps of the methods are described with reference to sequential series of reference signs and progression of the blocks in the figures, the method can be implemented in any particular order as desired.

1. A primary container assembly, comprising:
a primary container defining a body having a proximal end defining an opening configured to receive a plunger, a distal end opposite the proximal end and defining an outlet, and a chamber extending from the proximal end to the distal end that is configured to receive a drug;
a tube extending from a first end that is integrally attached to the distal end of the primary container to a second end opposite the first end, wherein the tube defines a channel extending from the first end to the second end; and
a needle configured to penetrate skin of a patient, wherein the needle is integrally attached to the second end of the tube,
wherein the tube is in fluid communication with the chamber of the primary container and the needle.

2. The primary container assembly of claim 1, wherein the tube comprises a flexible material.

3. The primary container assembly of claim 1, wherein the tube comprises a rigid, inflexible material.

4. The primary container assembly of claim 1, wherein the distal end of the primary container comprises a Luer connection integrally attached to the first end of the tube.

5. The primary container assembly of claim 1, further comprising:
a needle hub defining an interface between the needle and the tube.

6. The primary container assembly of claim 5, further comprising:
a needle shield disposed over at least a portion of the needle and releasably connected to the needle hub so as to maintain the needle in a sterile condition.

7. The primary container assembly of claim 6, wherein the needle shield is frictionally connected to the needle hub.

8. The primary container assembly of claim 6, further comprising:
a cup attached to the distal end of the primary container,
wherein the tube is configured to be placed in an initial coiled configuration, wherein a portion of the tube is coiled around a portion of the distal end of the primary container and substantially disposed within the cup.

9. The primary container assembly of claim 8, wherein the cup is releasably attached to the body of the primary container.

10. The primary container assembly of claim 8, wherein the needle shield is releasably secured to the cup.

11. The primary container assembly of claim 8, wherein when the needle shield is detached from the cup, the tube is configured to be transitioned from the coiled configuration to an uncoiled configuration, wherein the tube is uncoiled from around the portion of the distal end of the primary container.

12. The primary container assembly of claim 8, wherein the cup is a first cup, the primary container assembly further comprising:
a second cup releasably attached to the first cup, wherein the needle shield is substantially received within the second cup such that the tube maintains a concentric shape,
wherein the second cup is configured to interface with a filling machine.

13. The primary container assembly of claim 1, wherein the needle is a first needle, the primary container assembly further comprising:
a second needle defining an interface between the distal end of the body of the primary container and the first end of the tube.

14. The primary container assembly of claim 1, wherein the primary container and the tube are monolithic.

15. The primary container assembly of claim 1, wherein the tube defines a first length measured along an axis coaxial with a body of the tube from the first end to the second end, and the needle defines a second length, wherein the first length is greater than the second length.

16. A drug dispensing device, comprising:
the primary container assembly according to claim 1, wherein the primary container assembly further comprises a needle hub defining an interface between the needle and the tube; and
an input configured to engage the needle hub such that the tube is in fluid communication with the input.

17. A method for assembling a primary container assembly, comprising:
providing a primary container defining a body having a proximal end defining an opening configured to receive a plunger, a distal end opposite the proximal end and defining an outlet, and a chamber extending from the proximal end to the distal end that is configured to receive a drug;
attaching a first end of a tube to the distal end of the primary container, such that the tube is in fluid communication with the outlet; and
attaching a needle to a second end of the tube.

18. The method of claim 17, wherein attaching the tube to the primary container comprises molding the distal end of the primary container over the first end of the tube.

19. The method of claim 17, wherein attaching the tube to the primary container comprises monolithically forming the tube and the primary container.

20. The method of claim 17, further comprising:
filling the primary container with the drug.
